(19) United States
(12) Patent Application Publication
(10) Pub. No.: US 2005/0159775 A1
(43) Pub. Date: Jul. 21, 2005

(54) Disposable eyelid retractor apparatus

(76) Inventor: James W. Reynolds, Bryn Mawr, PA (US)

Correspondence Address: S. Michael Bender, P.O. Box 530-399, St. Petersburg, FL, 33747, US

(21) Appl. No.: 11/038,648

(22) Filed: Jan. 19, 2005

Related U.S. Application Data
(60) Provisional application No. 60/537,420, filed on Jan. 20, 2004.

(51) Int. Cl. A61B 17/44

(52) U.S. Cl. 606/205

(57) ABSTRACT

A disposable eyelid retractor apparatus includes an eyelid-adhering portion which includes a flexible substrate layer, a quantity of adhesive fixed on the flexible substrate layer, and a release strip attached to the adhesive. A retracting tab is connected to the flexible substrate layer by a flexible hinge, and a release tab is connected to an end portion of the flexible substrate layer. The disposable eyelid retractor apparatus can be used by a caregiver on another person to aid in the administration of ocular medication. In addition, the apparatus can be used by a person on one's own eye to self-administer ocular medication. Preferably, the flexible substrate layer and the release tab are formed as a unified structure. Even more preferably, the flexible substrate layer, the retracting tab, the flexible hinge, and the release tab are formed as a unified structure.

### CROSS-REFERENCE TO RELATED APPLICATION

This application claims priority based upon my copending Provisional Application Ser. No. 60/537,420, filed Jan. 20, 2004.

### BACKGROUND OF THE INVENTION

1. Field of the Invention

The present invention relates generally to devices for separating tissues in a human body, and more especially to devices especially adapted for separating a first eyelid from a second eyelid.

2. Description of the Prior Art

Devices are known in the prior art for separating a first eyelid from a second eyelid. For example, each of U.S. Pat. No. 5,433,190 and 6,440,065 discloses an eyelid retractor that has two arms for keeping a first eyelid separated from a second eyelid in a hands-free manner. This device is to be used by an ocular professional in conducting a professional ocular procedure. With each device, a first arm of the respective device has an end for engaging a first eyelid, and a second arm of the respective device has an end for engaging a second eyelid. Such devices are designed to be used by an ocular professional so that the ocular professional can conduct an ocular procedure without using one's hands to keep the eyelids in a retracted condition. Such devices cannot be used practically by a person to self-administer an ocular medication. It is well known that it is often difficult for a person to self-administer an ocular medication. So, in this respect, it would be desirable if a device were provided which facilitates self-administration of an ocular medication. In addition, such devices cannot be readily used by nonprofessional persons, such as child care personnel, in administering ocular medications to children in their care. In this respect, it would be desirable if a device were provided which facilitates administration of an ocular medication by nonprofessional persons, such as child care personnel.

U.S. Pat. No. 4,432,347 may be of interest for a disclosure of a cosmetic adhesive tape which can be used to stretch loose skin to correct the condition of a drooping eyelid. Such a cosmetic tape is not used to retract or separate a first eyelid from a second eyelid. The adhesive tape, in order to perform its function, does not have to come into contact with the eyelid margins or the eyeball. It is noted that with the devices in U.S. Pat. Nos. 5,433,190 and 6,440,065, such contact the eyelid margins or the eyeball can cause irritation or injury if not done in a careful and professional manner. In this respect, it would be desirable if an adhesive material could be employed in the retraction or separation of a first eyelid from a second eyelid, whereby the device bringing about the retraction does not contact the eyelid margins or the eyeball.

It is further noted that with the device in U.S. Pat. No. 4,432,347, the lack of retraction of the first eyelid from the second eyelid precludes the device from being used in the administration of an ocular medication. In this respect, it would be desirable if an adhesive-containing device were provided that facilitated the administration of an ocular medication.

U.S. Pat. No. 4,621,619 discloses a retractor that employs a hook for engaging a patient's skin in order to bring about retraction. The presence of a retracting hook would injure delicate eyelid margins and eyeballs. In this respect, it would be desirable to provide an eyelid retractor that does not employ hooks for engaging delicate tissues.

U.S. Pat. No. 5,213,114 may be of interest for its disclosure of a drape used for ocular procedures.

Still other features would be desirable in a disposable eyelid retractor apparatus. For example, once an ocular retractor is used, rather than carrying out time-consuming and expensive procedures for cleaning it for another use, it would be desirable if an ocular retractor were readily and economically disposable.

Thus, while the foregoing body of prior art indicates it to be well known to use ocular retractors, the prior art described above does not teach or suggest a disposable eyelid retractor apparatus which has the following combination of desirable features: (1) facilitates self-administration of an ocular medication; (2) facilitates administration of an ocular medication by nonprofessional persons, such as child care personnel; (3) employs an adhesive material in the retraction or separation of a first eyelid from a second eyelid, whereby the device bringing about the retraction does not contact the eyelid margins or the eyeball; (4) provides an adhesive-containing device that facilitates the administration of an ocular medication; (5) does not employ hooks for engaging delicate ocular tissues; and (6) provides an ocular retractor that is readily and economically disposable. The foregoing desired characteristics are provided by the unique disposable eyelid retractor apparatus of the present invention as will be made apparent from the following description thereof. Other advantages of the present invention over the prior art also will be rendered evident.

### SUMMARY OF THE INVENTION

To achieve the foregoing and other advantages, the present invention, briefly described, provides a disposable eyelid retractor apparatus which includes an eyelid-adhering portion which includes a flexible substrate layer, a quantity of adhesive fixed on the flexible substrate layer, and a release strip attached to the adhesive. A retracting tab is connected to the flexible substrate layer by a flexible hinge, and a release tab is connected to the flexible substrate layer.

The disposable eyelid retractor apparatus can be used by a caregiver on another person to aid in the administration of ocular medication. In addition, the apparatus can be used by a person on one's own eye to self-administer ocular medication.

Preferably, the release tab is connected to an end portion of the flexible substrate layer. Preferably, the flexible substrate layer and the release tab are formed as a unified structure. Even more preferably, the flexible substrate layer, the retracting tab, the flexible hinge, and the release tab are formed as a unified structure.

In accordance with another aspect of the invention, a method is provided of retracting a first eyelid from a second eyelid which includes the steps of:
obtaining an adhesive-layer containing hand-held member,
applying the adhesive layer to the first eyelid, whereby the adhesive-layer containing hand-held member is adhered to the first eyelid,
pulling on the hand-held member, whereby the first eyelid is separated from the second eyelid, and
separating the adhesive layer from the first eyelid, whereby the first eyelid is no longer separated from the second eyelid.

In accordance with still another aspect of the invention, a method is provided of applying a medicament to an eye which has a first eyelid and a second eyelid which includes the steps of:
obtaining an adhesive-layer containing hand-held member,
applying the adhesive layer to the first eyelid, whereby the adhesive-layer containing hand-held member is adhered to the first eyelid,
pulling on the hand-held member, whereby the first eyelid is retracted from the second eyelid,
applying the medicament to the eye between the retracted first and second eyelids, and
separating the adhesive layer from the first eyelid, whereby the first eyelid is no longer separated from the second eyelid.

The above brief description sets forth rather broadly the more important features of the present invention in order that the detailed description thereof that follows may be better understood, and in order that the present contributions to the art may be better appreciated. There are, of course, additional features of the invention that will be described hereinafter and which will be for the subject matter of the claims appended hereto.

In this respect, before explaining a preferred embodiment of the invention in detail, it is understood that the invention is not limited in its application to the details of the construction and to the arrangements of the components set forth in the following description or illustrated in the drawings. The invention is capable of other embodiments and of being practiced and carried out in various ways. Also, it is to be understood, that the phraseology and terminology employed herein are for the purpose of description and should not be regarded as limiting.

As such, those skilled in the art will appreciate that the conception, upon which disclosure is based, may readily be utilized as a basis for designing other structures, methods, and systems for carrying out the several purposes of the present invention. It is important, therefore, that the claims be regarded as including such equivalent constructions insofar as they do not depart from the spirit and scope of the present invention.

It is therefore an object of the present invention to provide a new and improved disposable eyelid retractor apparatus which has all of the advantages of the prior art and none of the disadvantages.

It is another object of the present invention to provide a new and improved disposable eyelid retractor apparatus which may be easily and efficiently manufactured and marketed.

It is a further object of the present invention to provide a new and improved disposable eyelid retractor apparatus which is of durable and reliable construction.

An even further object of the present invention is to provide a new and improved disposable eyelid retractor apparatus which is susceptible of a low cost of manufacture with regard to both materials and labor, and which accordingly is then susceptible of low prices of sale to the consuming public, thereby making such disposable eyelid retractor apparatus available to the buying public.

Still yet a further object of the present invention is to provide a new and improved disposable eyelid retractor apparatus which facilitates self-administration of an ocular medication.

Still another object of the present invention is to provide a new and improved disposable eyelid retractor apparatus that facilitates administration of an ocular medication by nonprofessional persons, such as child care personnel.

Yet another object of the present invention is to provide a new and improved disposable eyelid retractor apparatus which employs an adhesive material in the retraction or separation of a first eyelid from a second eyelid, whereby the device bringing about the retraction does not contact the eyelid margins or the eyeball.

Even another object of the present invention is to provide a new and improved disposable eyelid retractor apparatus that provides an adhesive-containing device that facilitates the administration of an ocular medication.

Still a further object of the present invention is to provide a new and improved disposable eyelid retractor apparatus which does not employ hooks for engaging delicate ocular tissues.

Yet another object of the present invention is to provide a new and improved disposable eyelid retractor apparatus that provides an ocular retractor that is readily and economically disposable.

These together with still other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there are illustrated preferred embodiments of the invention.

### BRIEF DESCRIPTION OF THE DRAWINGS

The invention will be better understood and the above objects as well as objects other than those set forth above will become more apparent after a study of the following detailed description thereof. Such description makes reference to the annexed drawing wherein:

FIG. 1 is a top view showing a preferred embodiment of the disposable eyelid retractor apparatus of the invention.

FIG. 2 is a side edge view of the embodiment of the disposable eyelid retractor apparatus shown in FIG. 1 taken along line 2-2 of FIG. 1.

FIG. 3 is a perspective view of the embodiment of the disposable eyelid retractor apparatus of FIG. 1, with the apparatus standing on a side edge.

FIG. 4 is a bottom view of the embodiment of the invention shown in FIG. 3 taken along line 4-4 thereof, wherein the release strip is partially removed from the eyelid-adhering portion.

FIG. 5 is a top view illustrating the adherence of the disposable eyelid retractor apparatus of the invention onto the top of a closed eyelid.

FIG. 6 is a top view illustrating a person grasping the retracting tab between a finger and a thumb and pulling upward so as to lift the eyelid off of the eyeball.

FIG. 7 illustrates removing the apparatus from the closed eyelid by the person grasping the release tab and pulling the apparatus off of the eyelid.

FIG. 8 illustrates the used disposable eyelid retractor apparatus of the invention being thrown into a wastebasket.

### DESCRIPTION OF THE PREFERRED EMBODIMENT

With reference to the drawings, a new and improved disposable eyelid retractor apparatus embodying the principles and concepts of the present invention will be described.

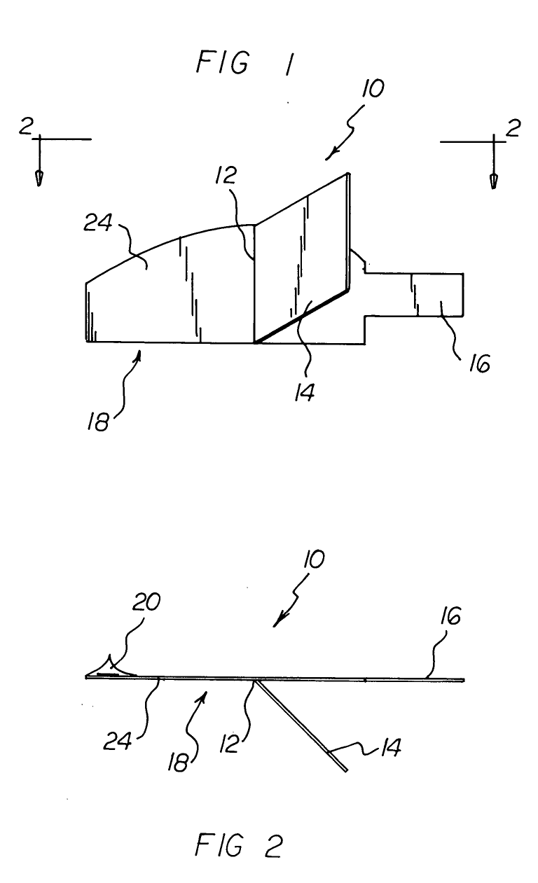

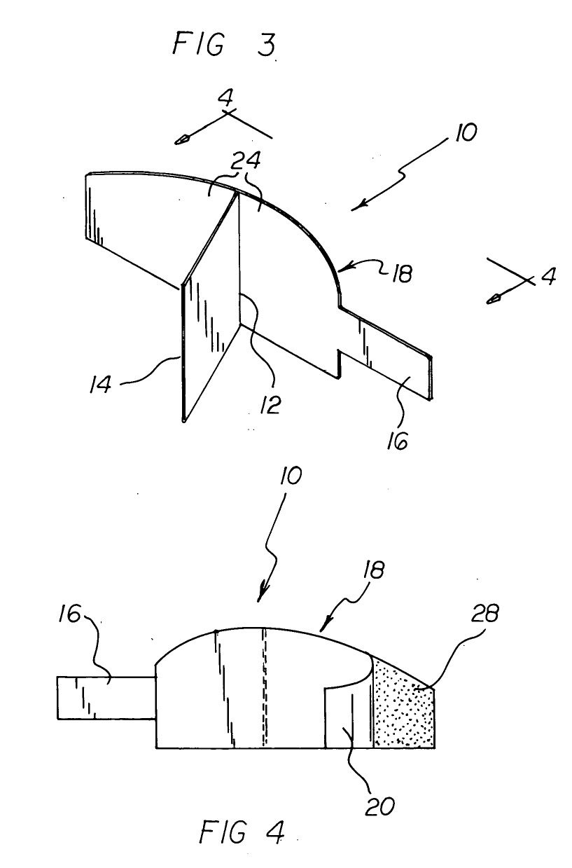

Turning to FIGS. 1-8, there is shown a preferred embodiment of the disposable eyelid retractor apparatus of the invention generally designated by reference numeral 10. In the preferred embodiment, disposable eyelid retractor apparatus 10 includes an eyelid-adhering portion 18 which includes a flexible substrate layer 24, a quantity of adhesive 28 fixed on a bottom side of the flexible substrate layer 24, and a release strip 20 attached to and covers the adhesive 28. A retracting tab 14 is connected to the flexible substrate layer 24 by a flexible hinge 12, and a release tab 16 is connected to the flexible substrate layer 24.

The disposable eyelid retractor apparatus 10 can be used by a caregiver on another person to aid in the administration of ocular medication. In addition, the apparatus can be used by a person on one's own eye to self-administer ocular medication.

Preferably, the release tab 16 is connected to an end portion of the flexible substrate layer 24. Preferably, the flexible substrate layer 24 and the release tab 16 are formed as a unified structure. Even more preferably, the flexible substrate layer 24, the retracting tab 14, the flexible hinge 12, and the release tab 16 are formed as a unified structure.

Preferably, the adhesive 28 is a hypoallergenic or non-allergenic biocompatible adhesive.

To use the disposable eyelid retractor apparatus 10 of the invention, the release strip 20 is first separated from the adhesive 28, as shown in FIG. 4. In this way, the adhesive 28 is exposed for contact with the eyelid 11.

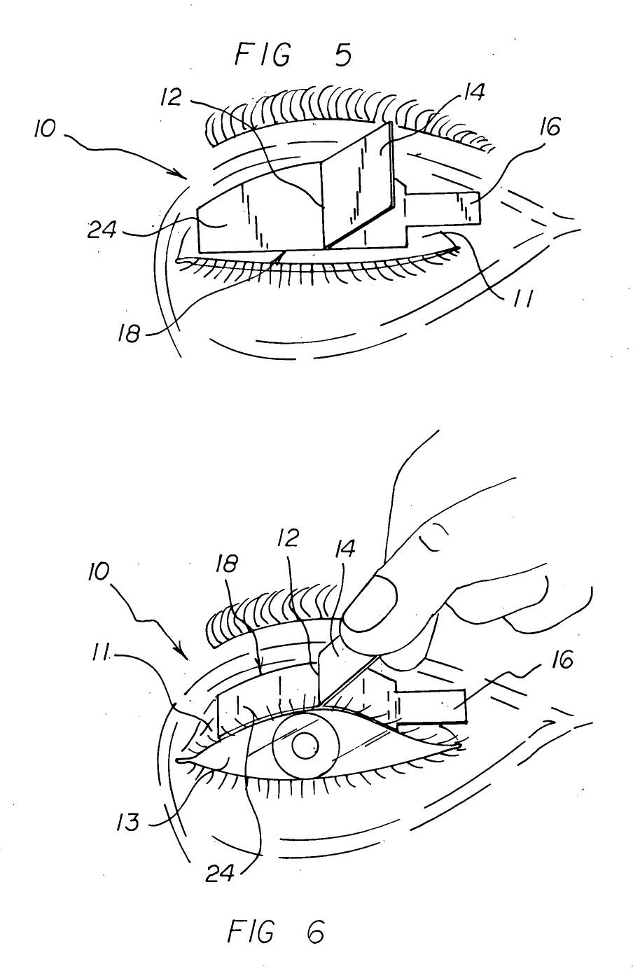

Then, as shown in FIG. 5, a patient closes an eyelid 11, and the adhesive 28 on the flexible substrate layer 24 is placed on the closed eyelid 11. The person applying the disposable eyelid retractor apparatus 10 to the eyelid 11 can grasp the retracting tab 14 to stabilize the applying of the apparatus to the eyelid 11.

Then, as shown in FIG. 6, a person grasps the retracting tab 14 and pulls upward on the retracting tab 14, thereby opening the closed eyelid 11. The retracting tab 14 can also be used to lift the eyelid 11 off of the eyeball 13. With the eyelid 11 in the open or raised position, it is easy to apply an agent to the eyelid margins and the eyeball 13. The applied agent can be in the form of drops, a gel, or an ointment.

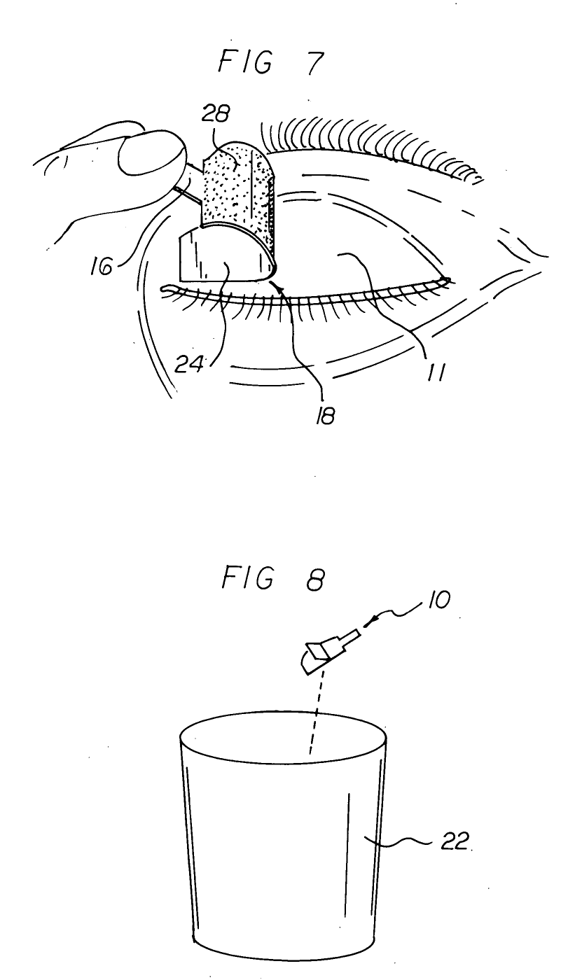

Once the agent has been applied to the eye, the eyelid 11 is closed, and a person can grasp the release tab 16 and pull up and back on the release tab 16, as shown in FIG. 7, to peel the eyelid-adhering portion 18 off of the closed eyelid 11.

After the disposable eyelid retractor apparatus 10 has been peeled off of the eyelid 11, the disposable eyelid retractor apparatus 10 can be disposed of in the wastebasket 22 as shown in FIG. 8.

The disposable eyelid retractor apparatus 10 is especially useful for treating eye conditions in children. For example, conjunctivitis is common in childhood, spreads easily amongst school-aged children, and at times can reach epidemic proportions. In this respect, most daycare centers and schools have a policy to deny access to any child with current eye infection, unless the infection is treated. This infection is treated with the application of topical antibiotic eyedrops, usually, four times a day for approximately one week.

It has never been easy for caregivers to administer the medications as ordered because it is generally difficult to get adequate compliance from a child. As a result, treatment failures or a delay in the resolution of the problem often occurs because of the inability of parents or caregivers to get prescribed medications into the eye and this may necessitate return visits to the physician's office.

The disposable eyelid retractor apparatus 10 of the invention permits a layperson to easily retract the eyelid 11 of a child so that application of ocular medication can be applied to the child's eye effectively.

The disposable eyelid retractor apparatus 10 of the invention can also be used to lift an eyelid 11 to permit inspection under the lifted eyelid 11 for foreign matter.

Aside from being used by a caregiver to another person, the disposable eyelid retractor apparatus 10 of the invention can also be used by a person on one's own eye to administer ocular medication to one's own eye.

It will be appreciated that the disposable eyelid retractor apparatus 10 of the invention can be applied to either the upper eyelid or the lower eyelid. By applying the disposable eyelid retractor apparatus 10 to the lower eyelid, drops of medication can easily be administered into the conjunctival sac i.e. the space between the eyeball and the lid.

It will further be appreciated that the disposable eyelid retractor apparatus 10 may be used to assist in the installation of contact lenses and for intra-operative lid retraction during surgical procedures, as will occur to those of ordinary skill in the art.

In accordance with another aspect of the invention, a method is provided of retracting a first eyelid from a second eyelid which includes the steps of: obtaining an adhesive-layer containing hand-held member; applying the adhesive layer to the first eyelid, whereby the adhesive-layer containing hand-held member is adhered to the first eyelid; pulling on the hand-held member, whereby the first eyelid is separated from the second eyelid; and separating the adhesive layer from the first eyelid, whereby the first eyelid is no longer separated from the second eyelid.

In accordance with another aspect of the invention, a method is provided of applying a medicament to an eye which has a first eyelid and a second eyelid which includes the steps of: obtaining an adhesive-layer containing hand-held member; applying the adhesive layer to the first eyelid, whereby the adhesive-layer containing hand-held member is adhered to the first eyelid; pulling on the hand-held member, whereby the first eyelid is retracted from the second eyelid; applying the medicament to the eye between the retracted first and second eyelids; and separating the adhesive layer from the first eyelid, whereby the first eyelid is no longer separated from the second eyelid.

The components of the disposable eyelid retractor apparatus of the invention can be made from inexpensive and durable plastic materials.

As to the manner of usage and operation of the instant invention, the same is apparent from the above disclosure, and accordingly, no further discussion relative to the manner of usage and operation need be provided.

It is apparent from the above that the present invention accomplishes all of the objects set forth by providing a new and improved disposable eyelid retractor apparatus that is low in cost, relatively simple in design and operation, and which may advantageously be used to facilitate self-administration of an ocular medication. With the invention, a disposable eyelid retractor apparatus is provided which facilitates administration of an ocular medication by nonprofessional persons, such as child care personnel. With the invention, a disposable eyelid retractor apparatus is provided which employs an adhesive material in the retraction or separation of a first eyelid from a second eyelid, whereby the device bringing about the retraction does not contact the eyelid margins or the eyeball. With the invention, a disposable eyelid retractor apparatus provides an adhesive-containing device that facilitates the administration of an ocular medication. With the invention, a disposable eyelid retractor apparatus is provided which does not employ hooks for engaging delicate ocular tissues. With the invention, a disposable eyelid retractor apparatus provides an ocular retractor that is readily and economically disposable.

Thus, while the present invention has been shown in the drawings and fully described above with particularity and detail in connection with what is presently deemed to be the most practical and preferred embodiment(s) of the invention, it will be apparent to those of ordinary skill in the art that many modifications thereof may be made without departing from the principles and concepts set forth herein, including, but not limited to, variations in size, materials, shape, form, function and manner of operation, assembly and use.

Hence, the proper scope of the present invention should be determined only by the broadest interpretation of the appended claims so as to encompass all such modifications as well as all relationships equivalent to those illustrated in the drawings and described in the specification.

Finally, it will be appreciated that the purpose of the annexed Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. Accordingly, the Abstract is neither intended to define the invention or the application, which only is measured by the claims, nor is it intended to be limiting as to the scope of the invention in any way.

1. A disposable eyelid retractor apparatus, comprising:
an eyelid-adhering portion which includes a flexible substrate layer, a quantity of adhesive fixed on said flexible substrate layer, and a release strip attached to said adhesive,
a retracting tab connected to said flexible substrate layer by a flexible hinge, and
a release tab connected to said flexible substrate layer.

2. The apparatus of claim 1 wherein said release tab is connected to an end portion of said flexible substrate layer.

3. The apparatus of claim 1 wherein said flexible substrate layer and said release tab are formed as a unified structure.

4. The apparatus of claim 1 wherein said flexible substrate layer, said retracting tab, said flexible hinge, and said release tab are formed as a unified structure.

5. A method of retracting a first eyelid from a second eyelid, comprising the steps of:
obtaining an adhesive-layer containing hand-held member,
applying the adhesive layer to the first eyelid, whereby the adhesive-layer containing hand-held member is adhered to the first eyelid,
pulling on the hand-held member, whereby the first eyelid is separated from the second eyelid, and
separating the adhesive layer from the first eyelid, whereby the first eyelid is no longer separated from the second eyelid.

6. A method of applying a medicament to an eye which has a first eyelid and a second eyelid, comprising the steps of:
obtaining an adhesive-layer containing hand-held member,
applying the adhesive layer to the first eyelid, whereby the adhesive-layer containing hand-held member is adhered to the first eyelid,
pulling on the hand-held member, whereby the first eyelid is retracted from the second eyelid,
applying the medicament to the eye between the retracted first and second eyelids, and
separating the adhesive layer from the first eyelid, whereby the first eyelid is no longer separated from the second eyelid.
